(19) United States
(12) Patent Application Publication
(10) Pub. No.: US 2025/0325620 A1
(43) Pub. Date: Oct. 23, 2025

(54) METHODS AND COMPOSITIONS USING DICHROSTACHYS GLOMERATA TO IMPROVE AND OR REDUCE INSOMNIA, IMPROVE TOTAL SLEEP DURATION AND QUALITY, IMPROVE DEEP SLEEP DURATION AND TIME IN LIGHT SLEEP, IMPROVE SLEEP LATENCY, IMPROVE THE AMOUNT OF TIME AWAKE AND IMPROVE SLEEP EFFICIENCY, READINESS AND BALANCE IN MAMMALS

(71) Applicant: Shil Kothari, Fairfield, CA (US)

(72) Inventor: Shil Kothari, Fairfield, CA (US)

(21) Appl. No.: 18/644,054

(22) Filed: Apr. 23, 2024

(51) Int. Cl. A61K 36/48; A61P 25/20

(52) U.S. Cl. CPC A61K 36/48; A61P 25/20

(57) ABSTRACT

Methods and related compositions using Dichrostachys glomerata to provide a variety of health and wellness benefits in a mammal. More specifically, methods and compositions using Dichrostachys glomerata to improve and or reduce insomnia, improve total sleep duration and quality, improve deep sleep duration and time in light sleep, improve sleep latency, improve the amount of time awake and improve sleep efficiency, readiness and balance in a mammal.

### FIELD OF THE DISCLOSURE

The present disclosure relates to methods and related compositions using Dichrostachys glomerata to provide a variety of health and wellness benefits in a mammal. More specifically, the present disclosure is directed to various methods and compositions for using Dichrostachys glomerata to improve and or reduce insomnia, improve total sleep duration and quality, improve deep sleep duration and time in light sleep, improve sleep latency, improve the amount of time awake and improve sleep efficiency, readiness and balance in mammals.

### BACKGROUND

Quality sleep that is restful and restorative is essential for maintaining physical, social, and psychological well-being.¹⁻³ Insufficient or poor quality sleep are associated with adverse health outcomes including reduced productivity, impaired work performance, and lower levels of physical activity.^{4.5} Despite the well-established importance of sleep, sleep dissatisfaction is prevalent among the general population, with up to 41.7% of adults reporting insufficient sleep and 48% reporting difficulties with sleep initiation or maintenance.⁶ Although medication may be effective for treating sleep disorders, there is still an unmet need for safe and accessible sleep aids for individuals with suboptimal non-clinical sleep issues and individuals that want or need a sleep aid who efficacy does not lessen with longer term use and can be used long term.⁷

Furthermore, traditional interventions involving over-the-counter and prescription medications often come with undesirable side effects, limited efficacy that often deteriorates over time, and the potential for dependency and further complications.⁷ Therefore, as the results below and in the FIGS. indicate, research was conducted to determine whether alternative novel compositions containing Dichrostachys glomerata effect a variety of sleep and rest related factors. As the results shown below and in the FIGS. demonstrate, the compositions containing Dichrostachys glomerata improve and or reduce insomnia, improve total sleep duration and quality, improve deep sleep duration and time in light sleep, improve sleep latency, improve the amount of time awake and improve sleep efficiency, readiness and balance in mammals.

Despite the widespread use of herbal plants for addressing various health concerns, there are limited randomized controlled trials evaluating their effectiveness for improving sleep quality and various important sleep and rest related factors.⁸ Within this context, Dichrostachys glomerata, a plant known for its safety profile and longstanding use as a spice in African cooking for many decades if not centuries, is generally considered very safe for long term consumption. In regions like western Cameroon, the fruit and seeds of the Dichrostachys glomerata plant are commonly employed as a spice, reflecting its cultural relevance and accessibility.⁹

Dichrostachys glomerata is a semi-deciduous to deciduous tree that grows up to about 7 meters tall with an open crown. Bark on young branches typically appears green in color and hairy but dark grey-brown and longitudinally fissured on older branches and stems with smooth on spines formed from modified side shoots.

Dichrostachys glomerata boasts a rich chemical composition, featuring flavonoids, phenolic compounds, alkaloids, tannins, saponins, and terpenoids.¹⁰ Existing in vitro and in vivo research has found its antioxidant properties, along with its ability to reduce fasting serum glucose levels and glycated hemoglobin.¹¹⁻¹³ Moreover, Dichrostachys glomerata has been shown to effect cardiovascular diseases risk factors and have inflammatory, anthropometric and lipomodulatory effects.^{14,15}

Therefore, extensive experiments were performed to determine the effectiveness of a standardized powder derived from Dichrostachys glomerata fruit pods on a variety of sleep and rest related factors. The results of the randomized double-blind placebo-controlled trial designed experiments, shown in the FIGS. and discussed in detail below, demonstrated statistically significant improvement in insomnia, total sleep duration and quality, deep sleep duration and time in light sleep, sleep latency, the amount of time awake and sleep efficiency, readiness and balance in adults. The primary outcomes were self-reported and objective sleep quality or quantity. The secondary outcomes were daytime activity and safety or adverse events.

### SUMMARY OF THE INVENTION

In one embodiment of the present disclosure, an effective amount of a composition containing Dichrostachys glomerata is provided to a mammal to improve and or reduce insomnia, improve total sleep duration and quality, improve deep sleep duration and time in light sleep, improve sleep latency, improve the amount of time awake and improving sleep efficiency, readiness and balance in the mammal.

In one aspect of at least one embodiment of the present disclosure, the effective amount of a composition containing Dichrostachys glomerata is provided as an oral dosage unit in the form of a pill, capsule, liquid, lozenge or tablet or other known form.

The compositions and methods of the present disclosure can be provided, used or administered using any delivery method currently known or discovered in the future.

In another aspect of at least one embodiment of the present disclosure, the effective amount of Dichrostachys glomerata provided to the mammal is 100 mg to 900 mg per day.

In yet another aspect of at least one embodiment of the present disclosure, the composition is an aqueous extract of Dichrostachys glomerata.

In yet another aspect of at least one embodiment of the present disclosure, the composition is an ethanol extract of Dichrostachys glomerata.

In yet another aspect of at least one embodiment of the present disclosure, the composition is comprised of fruit pods of Dichrostachys glomerata.

In yet another aspect of at least one embodiment of the present disclosure, the composition is comprised of dried fruit pods of Dichrostachys glomerata.

In yet another aspect of at least one embodiment of the present disclosure, a method of improving and or reducing insomnia, improving total sleep duration and quality, improving deep sleep duration and time in light sleep, improving sleep latency, improving the amount of time awake and improving sleep efficiency, readiness and balance in mammal is provided. The method comprises providing or administering a composition containing an effective amount of Dichrostachys glomerata to a mammal.

In yet another aspect of at least one embodiment of the present disclosure, the method includes the composition being provided or administered to the mammal as an oral dosage unit in the form of a capsule, tablet or other known form or delivery method.

In yet another aspect of at least one embodiment of the present disclosure, the effective amount of the Dichrostachys glomerata composition provided or administered by the method is 100 mg to 900 mg daily for at least 30 days.

In yet another aspect of at least one embodiment of the present disclosure, the method comprises providing or administering an aqueous extract of the Dichrostachys glomerata composition.

In yet another aspect of at least one embodiment of the present disclosure, the method comprising providing or administering an ethanol extract of the Dichrostachys glomerata composition.

In yet another aspect of at least one embodiment of the present disclosure, the method comprises providing or administering a composition of Dichrostachys glomerata fruit pods to a mammal.

### BRIEF DESCRIPTION OF THE DRAWINGS

The foregoing and other objects, aspects, features, and advantages of the present disclosure will become more apparent and better understood by referring to the following description taken in conjunction with the accompanying drawings, in which:

FIG. 1 is a table showing Table 1: Mean (M) and Standard Deviation (SD) Scores for the Insomnia Severity Index and Oura Ring Sleep and Activity Data for the Dichrostachys glomerata Group and Placebo Group by Time. Note: Lower scores indicate an improvement for Insomnia Severity Index, Time Awake, and Sleep Latency. Higher scores indicate an improvement for Overall Sleep Score, Sleep Duration, Total Sleep Time, Activity Score, Stay Active Score, Readiness Sleep Balance Score, and Sleep Efficiency, and REM. Min=minutes.

FIG. 2 is a table showing Descriptive Statistics and Repeated-Measures ANOVA for the Self-Report Outcomes of the Insomnia Severity Index and Pittsburgh Sleep Quality Index. Interpretation: Lower scores indicate an improvement in insomnia symptoms. Significant main effects for time and condition was shown by the data obtained and shown herein. Insomnia symptoms decreased significantly from Baseline to Day 60 for the DygloFit (extract of Dichrostachys glomerata fruit pods) group of mammals. +=significant differences from Baseline to Day 60.

FIG. 3 is a table showing Descriptive Statistics and Repeated-Measures ANOVA for the Self-Report Outcomes of the Insomnia Severity Index and Pittsburgh Sleep Quality Index. Interpretation: Lower scores indicate an improvement in subjective sleep quality. Significant main effects for time and condition was shown by the data obtained and shown. Subjective sleep quality improved significantly from Baseline to Days 30 and 60 for the DygloFit (extract of Dichrostachys glomerata fruit) group and from Baseline to Day 60 for the Placebo group. *=significant differences from Baseline to Day 30. +=significant differences from Baseline to Day 60.

FIG. 4 is a table showing Descriptive Statistics and Repeated-Measures ANOVA for the Self-Report Outcomes of the Insomnia Severity Index and Pittsburgh Sleep Quality Index. Interpretation: Lower scores indicate an improvement in sleep latency. Significant main effects for time and condition, but no significant effect for interaction was shown by the data obtained and shown. Sleep latency improved significantly from Baseline to Days 30 and 60 for the Placebo group. *=significant differences from Baseline to Day 30. +=significant differences from Baseline to Day 60.

FIG. 5 is a table showing Descriptive Statistics and Repeated-Measures ANOVA for the Self-Report Outcomes of the Insomnia Severity Index and Pittsburgh Sleep Quality Index. Interpretation: Lower scores indicate an improvement in sleep duration. Significant main effects for time and condition, but no significant effect for interaction was shown by the data obtained and shown. Sleep duration improved significantly by showing it decreased significantly from Baseline to Day 60 for the Placebo group. *=significant differences from Baseline to Day 30. +=significant differences from Baseline to Day 60.

FIG. 6 is a table showing Descriptive Statistics and Repeated-Measures ANOVA For the Self-Report Outcomes of the Insomnia Severity Index and Pittsburgh Sleep Quality Index. Interpretation: Lower scores indicate an improvement in subjective sleep efficiency. No significant main effects for time, or interaction were obtained by the data obtained and shown. A significant condition effect was found with the data obtained showing the placebo having a larger improvement.

FIG. 7 is a table showing Descriptive Statistics and Repeated-Measures ANOVA for the Self-Report Outcomes of the Insomnia Severity Index and Pittsburgh Sleep Quality Index. Interpretation: Lower scores indicate an improvement in sleep disturbance. Significant main effects for time and condition, but no significant effect for interaction was shown by the data obtained. Sleep disturbance decreased significantly from Baseline to Day 60 for the DygloFit (extract of Dichrostachys glomerata fruit) group. *=significant differences from Baseline to Day 30. +=significant differences from Baseline to Day 60.

FIG. 8 is a table showing Descriptive Statistics and Repeated-Measures ANOVA for the Self-Report Outcomes of the Insomnia Severity Index and Pittsburgh Sleep Quality Index. Interpretation: Lower scores indicate an improvement in sleep medication use. Significant main effects for time and condition, but no significant effect for interaction was shown by the data obtained. Sleep medication use decreased significantly from Baseline to Days 30 and 60 for the Placebo group. *=significant differences from Baseline to Day 30. +=significant differences from Baseline to Day 60.

FIG. 9 is a table showing Descriptive Statistics and Repeated-Measures ANOVA for the Self-Report Outcomes of the Insomnia Severity Index and Pittsburgh Sleep Quality Index. Interpretation: Lower scores indicate an improvement in daytime dysfunction. No significant main effects for time or interaction were evidenced by the data obtained. A significant main effect for condition was evidenced indicating an improvement by Day 60 for the DygloFit (extract of Dichrostachys glomerata fruit) group.

FIG. 10 is a table showing Descriptive Statistics and Repeated-Measures ANOVA for the Self-Report Outcomes of the Insomnia Severity Index and Pittsburgh Sleep Quality Index. Interpretation: Lower scores indicate an improvement in global sleep score. Significant main effects for time and condition, but no significant effect for interaction was evidenced by the data obtained. Global sleep scores decreased significantly, indicating an improvement in global sleep, from Baseline to Day 60 for the DygloFit (extract of Dichrostachys glomerata fruit) Group and from Baseline to Days 30 and 60 for the Placebo group. *=significant differences from Baseline to Day 30. +=significant differences from Baseline to Day 60.

FIG. 11 is a table showing Descriptive Statistics Baseline—Week 4 Oura Sleep Data.

FIG. 12 is a table showing Descriptive Statistics Week 5—Week 8 Oura Sleep Data.

FIG. 13 is a table showing Repeated-Measures ANOVAs Oura Sleep Data. Interpretation: Ranging from 0 to 100, the Sleep Score is an overall measure of how well people sleep. 85 or over indicates optimal sleep quality; 70 to 84 indicates good overall sleep quality; under 70 indicates disturbed sleep quality. Pairwise comparisons show that there was a significant difference from Baseline to Week 8, Week 1 to Week 2, Week 2 to Week 8, Week 5 to Week 8, and Week 7 to Week 8 for the DygloFit (extract of Dichrostachys glomerata fruit) group. There were also significant differences at Weeks 3, 4, & 6 where the Placebo group outperformed the DygloFit (extract of Dichrostachys glomerata fruit) group. #=significant difference between DygloFit (extract of Dichrostachys glomerata fruit) group and Placebo group.

FIG. 14 is a table showing Repeated-Measures ANOVAs Oura Sleep Data. Interpretation: Represents circadian alignment's contribution for Sleep Score. Sleep midpoint time between 12 AM and 3 AM gives highest score. The more the midpoint time deviates from that range, the lower the score. Pairwise comparisons show that there was a significant difference from Baseline to Weeks 1, 2, 5, 7, & 8 for the DygloFit (extract of Dichrostachys glomerata fruit) group.

FIG. 15 is a table showing Repeated-Measures ANOVAs Oura Sleep Data. Interpretation: Higher scores indicate improved deep sleep. Pairwise comparisons show that there was a significant difference from Baseline to Weeks 3, 5, & 8, and Week 3 to Week 4 for the DygloFit (extract of Dichrostachys glomerata fruit) group, and from Baseline to Weeks 2, 4, & 8, Week 1 to Week 2, 4, 5, & 8, Week 2 to Week 7, and Week 7 to Week 8 for the Placebo group. There were also significant differences at Week 4 where the Placebo group outperformed the DygloFit (extract of Dichrostachys glomerata fruit) group, and Week 8 where the DygloFit (extract of Dichrostachys glomerata fruit) group outperformed the Placebo group. #=significant difference between DygloFit (extract of Dichrostachys glomerata fruit) group and Placebo group.

FIG. 16 is a table showing Repeated-Measures ANOVAs Oura Sleep Data. Interpretation: Lower scores indicate improvements in sleep disturbances. Pairwise comparisons show that there was a significant difference from Baseline to Weeks 3, 5, & 8, and Week 3 to Week 4 for the DygloFit (extract of Dichrostachys glomerata fruit) group, and from Baseline to Week 8 for the DygloFit (extract of Dichrostachys glomerata fruit) group and from Week 1 to Week 7, and Week 3 to Week 7 for the Placebo group. There were also significant differences at Weeks 7 & 8 where the Placebo group outperformed the DygloFit (extract of Dichrostachys glomerata fruit) group. #=significant difference between DygloFit (extract of Dichrostachys glomerata fruit) group and Placebo group.

FIG. 17 is a table showing Repeated-Measures ANOVAs Oura Sleep Data. Interpretation: Sleep Efficiency represents the percentage of time spent asleep compared to time spent awake while in bed, and it is calculated by dividing total sleep by sleep duration and multiplying by 100. Sleep efficiency of 85% is a sign of peaceful and uninterrupted sleep. Pairwise comparisons show that there was a significant difference from Baseline to Week 8, and Weeks 1-7 to Week 8 for the Dyglofit (extract of Dichrostachys glomerata fruit) group. There were also significant differences at Weeks 2, 4, & 5 where the Placebo group outperformed the DygloFit (extract of Dichrostachys glomerata fruit) group. #=significant difference between DygloFit (extract of Dichrostachys glomerata fruit) group and Placebo group.

FIG. 18 is a table showing Repeated-Measures ANOVAs Oura Sleep Data. Interpretation: Sleep latency is the amount of time that it takes to fall asleep at night. Falling asleep without extended delay (ideally within 15 to 20 minutes of trying to go to sleep) is often considered ideal. Falling asleep in less than 5 minutes can be a sign of overtiredness. Pairwise comparisons show that there was a significant difference from Baseline to Week 1, Week 1 to Week 2, and Week 2 to Weeks 6 & 7, for the DygloFit (extract of Dichrostachys glomerata fruit) group, and from Baseline to Week 8, Weeks 1, 2, 3, 5, & 7 to Week 8. There were also significant differences at Week 8 where the Placebo group outperformed the DygloFit (extract of Dichrostachys glomerata fruit) group. #=significant difference between DygloFit (extract of Dichrostachys glomerata fruit) group and Placebo group.

FIG. 19 is a table showing Repeated-Measures ANOVAs Oura Sleep Data. Interpretation: REM sleep is associated with dreaming, memory consolidation, and creativity. REM sleep plays an important role in re-energizing the mind and body. REM sleep makes up anywhere between 5 to 50% of total sleep time. On average, the optimal amount for healthy adults starts from 1.5 hours. Pairwise comparisons show that there was a significant difference from Baseline to Week 2, Week 2 to Week 7, and Week 4 to Week 5 for the DygloFit (extract of Dichrostachys glomerata fruit) group, and from Week 1 to Week 8, and Weeks 4 & 5 to Week 8for the Placebo group. There were also significant differences at Weeks 1, 2, 3, & 5 where the Placebo group outperformed the DygloFit (extract of Dichrostachys glomerata fruit) group. #=significant difference between DygloFit (extract of Dichrostachys glomerata fruit) group and Placebo group.

FIG. 20 is a table showing Repeated-Measures ANOVAs Oura Sleep Data. Interpretation: Represents total sleep time's contribution for sleep quality. The value depends on age of the user—the younger, the more sleep is needed for good score. Pairwise comparisons show that there was a significant difference from Baseline to Week 2, and Week 2 to Weeks 4, 5, & 8 for the DygloFit (extract of Dichrostachys glomerata fruit) group. There were also significant differences at Weeks 1, 2, 3, & 6 where the Placebo group outperformed the DygloFit (extract of Dichrostachys glomerata fruit) group. #=significant difference between DygloFit (extract of Dichrostachys glomerata fruit) group and Placebo group.

FIG. 21 is a table showing Repeated-Measures ANOVAs Oura Sleep Data. Interpretation: Higher score indicates a longer sleep duration. Pairwise comparisons show that there were significant differences at Weeks 1, 2, 3, & 5 where the Placebo group outperformed the DygloFit (extract of Dichrostachys glomerata fruit) group. #=significant difference between DygloFit (extract of Dichrostachys glomerata fruit) group and Placebo group.

FIG. 22 is a table showing Repeated-Measures ANOVAs Oura Sleep Data. Interpretation: Awake time is the time spent awake in bed before and after falling asleep. Pairwise comparisons show that there was a significant difference from Baseline to Week 8, and Weeks 1-7 to Week 8 for the DygloFit (extract of Dichrostachys glomerata fruit) group. There were also significant differences at Week 5 where the Placebo group outperformed the DygloFit (extract of Dichrostachys glomerata fruit) group. #=significant difference between DygloFit (extract of Dichrostachys glomerata fruit) group and Placebo group.

FIG. 23 is a table showing Repeated-Measures ANOVAs Oura Sleep Data. Interpretation: Light sleep should account for between 45-55% of a healthy and natural sleep cycle, therefore increased amounts of Light sleep indicate less time in restorative stages of REM and Deep sleep. Pairwise comparisons show that there was a significant difference from Week 3 to Week 4 for the DygloFit (extract of Dichrostachys glomerata fruit) group. There were also significant differences at Weeks 1-8 where the DygloFit (extract of Dichrostachys glomerata fruit) group outperformed the Placebo group. #=significant difference between DygloFit (extract of Dichrostachys glomerata fruit) group and Placebo group.

FIG. 24 a table showing Repeated-Measures ANOVAs Oura Sleep Data. Interpretation: REM sleep is associated with dreaming, memory consolidation, and creativity. REM sleep plays an important role in re-energizing the mind and body. REM sleep makes up anywhere between 5 to 50% of total sleep time. On average, the optimal amount for healthy adults starts from 1.5 hours. Pairwise comparisons show that there was a significant difference from Baseline to Week 5 for the DygloFit (extract of Dichrostachys glomerata fruit) group, and from Baseline to Week 8, and Week 1 to Week 8 for the Placebo group. There were also significant differences at Weeks 1-5 where the Placebo group outperformed the DygloFit (extract of Dichrostachys glomerata fruit) group. #=significant difference between DygloFit (extract of Dichrostachys glomerata fruit) group and Placebo group.

FIG. 25 is a table showing Repeated-Measures ANOVAs Oura Sleep Data. Interpretation: Deep sleep is the most restorative and rejuvenating sleep stage. Deep sleep makes up anywhere from 0 to 35% of total sleep. On average adults spend 15-20% (1 to 1.5 hours) of their total sleep time in deep sleep. Pairwise comparisons show that there was a significant difference from Baseline to Week 8 for the DygloFit (extract of Dichrostachys glomerata fruit) group, and from Baseline to Weeks 2, 3, 5, & 8 for the Placebo group. There were also significant differences at all Weeks where the DygloFit (extract of Dichrostachys glomerata fruit) group outperformed the Placebo group. #=significant difference between DygloFit (extract of Dichrostachys glomerata fruit) group and Placebo group.

FIG. 26 is a table showing Repeated-Measures ANOVAs Oura Sleep Data. Interpretation: Total sleep reflects the amount of time spent in light, REM, and deep sleep. Pairwise comparisons show that there were significant differences at 1, 2, 3, & 5 Week where the Placebo group outperformed the DygloFit (extract of Dichrostachys glomerata fruit) group. #=significant difference between DygloFit (extract of Dichrostachys glomerata fruit) group and Placebo group.

FIG. 27 is a table showing Repeated-Measures ANOVAs Oura Sleep Data. Interpretation: Sleep latency is the amount of time that it takes to fall asleep at night. Falling asleep in less than 5 minutes can be a sign of overtiredness. Pairwise comparisons show that there was a significant difference from Week 3 to Weeks 4-6, Week 4 to Week 8, Week 5 to Week 8, and Week 6 to Week 8 for the DygloFit (extract of Dichrostachys glomerata fruit) group.

FIG. 28 is a table showing Repeated-Measures ANOVAs Oura Sleep Data. Interpretation: Sleep efficiency is the percentage of the sleep period spent asleep. Pairwise comparisons show that there was a significant difference from Baseline to Week 8, Weeks 1-7 to Week 8 for the DygloFit (extract of Dichrostachys glomerata fruit) group. There were also significant differences at Weeks 1, 4, & 5 where the Placebo group outperformed the DygloFit (extract of Dichrostachys glomerata fruit) group. #=significant difference between DygloFit (extract of Dichrostachys glomerata fruit) group and Placebo group.

FIG. 29 is a table showing Repeated-Measures ANOVAs Oura Sleep Data. Interpretation: Percentage of sleep time when the user was moving, where a higher value is indicative of increased movement during sleep. Pairwise comparisons show that there was a significant difference from Week 1 to Week 7 for the Placebo group. There were also significant differences at Weeks 7 & 8 where the DygloFit (extract of Dichrostachys glomerata fruit) group outperformed the Placebo group. #=significant difference between DygloFit (extract of Dichrostachys glomerata fruit) group and Placebo group.

FIG. 30 is a table showing Repeated-Measures ANOVAs Oura Sleep Data. Interpretation: The number of time the user woke up from bed during the sleep period. Pairwise comparisons show that there was a significant difference from Baseline to Weeks 2, 3, 6, & 8 for the DygloFit (extract of Dichrostachys glomerata fruit) group, and from Week 3 to Week 7 the Placebo group.

FIG. 31 is a table showing Descriptive Statistics Baseline—Week 4 Oura Activity Data.

FIG. 32 is a table showing Descriptive Statistics Week 5—Week 8 Oura Activity Data.

FIG. 33 is a table showing Repeated-Measures ANOVAs Oura Activity Data. Interpretation: Activity scores take into account how ready you are for the day, your sleep the previous night, and the balance between activity, inactivity and rest; higher scores indicates an achievement of optimal balance between activity and rest, with scores of ≥85 indicating optimal balance. Pairwise comparisons show that there was a significant difference from Baseline to Weeks 7, & 8, and Week 1 to Week 7 for the DygloFit (extract of Dichrostachys glomerata fruit) group, and from Baseline to Weeks 7 & 8, and Week 2 to Weeks 3, 7, & 8 for the Placebo group. There were also significant differences at Weeks 3, 4, 7 & 8 where the DygloFit (extract of Dichrostachys glomerata fruit) group outperformed the Placebo group. #=significant difference between DygloFit (extract of Dichrostachys glomerata fruit) group and Placebo group.

FIG. 34 is a table showing Repeated-Measures ANOVAs Oura Activity Data. Interpretation: This Activity Score contributor indicates how well the ring user has managed to avoid inactivity (sitting or standing still) during last 24 hours. The more inactivity, the lower contributor value. The contributor value is 100 when inactive time during past 24 hours is below 5 hours. The contributor value is above 95 when inactive time during past 24 hours is below 7 hours. There were no significant differences on pairwise comparisons.

FIG. 35 is a table showing Repeated-Measures ANOVAs Oura Activity Data. Interpretation: This Activity Score contributor indicates how often the ring user has reached his/her daily activity target during seven last days. Pairwise comparisons show that there was a significant difference from Baseline to Weeks 7, & 8, and Week 1 to Week 7 for the DygloFit (extract of Dichrostachys glomerata fruit) group, and from Baseline to Week 8, Week 1 to Weeks 2, 3, 4, & 7, Week 4 to Week 5, Week 5 to Week 8, and Week 6 to Week 8, and from Baseline to Weeks 1, 4, 5, 6, & 7 for the Placebo group. There were also significant differences at Weeks 1, 5, & 6 where the Placebo group outperformed the DygloFit (extract of Dichrostachys glomerata fruit) group. #=significant difference between DygloFit (extract of Dichrostachys glomerata fruit) group and Placebo group.

FIG. 36 is a table showing Repeated-Measures ANOVAs Oura Activity Data. Interpretation: Daily physical activity converted into approximate meters i.e. amount of walking needed to get the same amount of activity. Pairwise comparisons show that there was a significant difference at Weeks 2 & 7 where the DygloFit (extract of Dichrostachys glomerata fruit) group outperformed the Placebo group. #=significant difference between DygloFit (extract of Dichrostachys glomerata fruit) group and Placebo group.

FIG. 37 is a table showing Repeated-Measures ANOVAs Oura Activity Data. Interpretation: Activity scores take into account how ready you are for the day, your sleep the previous night, and the balance between activity, inactivity and rest; higher scores indicates an achievement of optimal balance between activity and rest, with scores of ≥85 indicating optimal balance. Pairwise comparisons show that there was a significant difference from Week 1 to Weeks 2, 3, & 4 for the DygloFit (extract of Dichrostachys glomerata fruit) group, and from Week 1 to Week 8, and Week 6 to Week 8 for the Placebo group.

FIG. 38 is a table showing Descriptive Statistics Baseline—Week 4 Oura Readiness Data.

FIG. 39 is a table showing Descriptive Statistics Week 5—Week 8 Oura Readiness Data.

FIG. 40 is a table showing Repeated-Measures ANOVAs Oura Readiness Data. Interpretation: Activity Balance measures how your activity level over the past 14 days (with the past 2-5 being weighted slightly more) is affecting the persons readiness to perform. When your activity balance is optimal, it typical means that the person tested has been active, but kept from training at their maximum capacity. This has boosted their recovery and helped build up their energy levels. Higher scores indicate more readiness. Pairwise comparisons show that there was a significant difference from Baseline to Week 6, and Week 1 to Weeks 2, 3, & 4, Week 2 to Week 6, Week 3 to Weeks 5 & 6, Week 4 to Week 6, Week 5 to Week 8, Week 6 to Weeks 7 & 8, and Week 7 to Week 8 for the DygloFit (extract of Dichrostachys glomerata fruit) group, and from Week 4 to Week 8 for the Placebo group. There were also significant differences at Weeks 6 & 8 where the DygloFit (extract of Dichrostachys glomerata fruit) group outperformed the Placebo group. #=significant difference between DygloFit (extract of Dichrostachys glomerata fruit) group and Placebo group.

FIG. 41 is a table showing Repeated-Measures ANOVAs Oura Readiness Data. Interpretation: Measurement of recovery status. Oura interprets a mostly high HRV with some lowered HRV due to training or other healthy activities to be the optimal HRV balance. Pairwise comparisons show that there was a significant difference from Baseline to Weeks 5 & 6, Week 1 to Week 8, Week 2 to Weeks 5 & 6, Week 3 to Week 8, Week 4 to Week 6, and Weeks 5-7 to Week 8 for the DygloFit (extract of Dichrostachys glomerata fruit) group, and from Baseline to Weeks 5-7, Week 1 to Weeks 3-8, and Week 2 to Weeks 5-8 for the Placebo group. There were also significant differences at Week 1 where the DygloFit (extract of Dichrostachys glomerata fruit) group outperformed the Placebo group. #=significant difference between DygloFit (extract of Dichrostachys glomerata fruit) group and Placebo group.

FIG. 42 is a table showing Repeated-Measures ANOVAs Oura Readiness Data. Interpretation: Sleep Balance compares how well you have been sleeping in the past 14 days against your long-term average, with the past few nights of sleep weighing more heavily on your score. In other words, if you slept poorly last night, this will impact your Sleep Balance more in comparison to a poor night of sleep that occurred one week ago. This is designed to serve as a call-out for when you may need to make your sleep for the upcoming night more of an immediate priority before sleep debt begins to accumulate. Pairwise comparisons show that there was a significant difference from Baseline to Weeks 2 & 3, Week 1 to Week 8, Week 2 to Weeks 4, 6, & 8, Week 3 to Weeks 4, 5, 6, & 8, Week 5 to Week 8, and Week 7 to Week 8 for the DygloFit (extract of Dichrostachys glomerata fruit) group, and from Baseline to Weeks 3 & 4, Week 1 to Weeks 3 & 4, Week 3 to Weeks 5, 7, & 8, and Week 4 to Weeks 7 & 8 for the Placebo group. There were also significant differences at Week 3 where the Placebo group outperformed the DygloFit (extract of Dichrostachys glomerata fruit) group and at Week 8 where the DygloFit (extract of Dichrostachys glomerata fruit) group outperformed the Placebo group. #=significant difference between DygloFit (extract of Dichrostachys glomerata fruit) group and Placebo group.

FIG. 43 is a table showing Descriptive Statistics Baseline to Week 4 Sleep Survey Results. For self-report assessments higher scores indicate an improvement.

FIG. 44 is a table showing Descriptive Statistics Weeks 5 to 8 Sleep Surveys Results.

FIG. 45 is a table showing Repeated-Measures ANOVAs Sleep. Interpretation: Higher scores indicate an improvement. Pairwise comparisons show that there were significant differences for the DygloFit (extract of Dichrostachys glomerata fruit) group. #=significant difference between Placebo group and DygloFit (extract of Dichrostachys glomerata fruit) group.

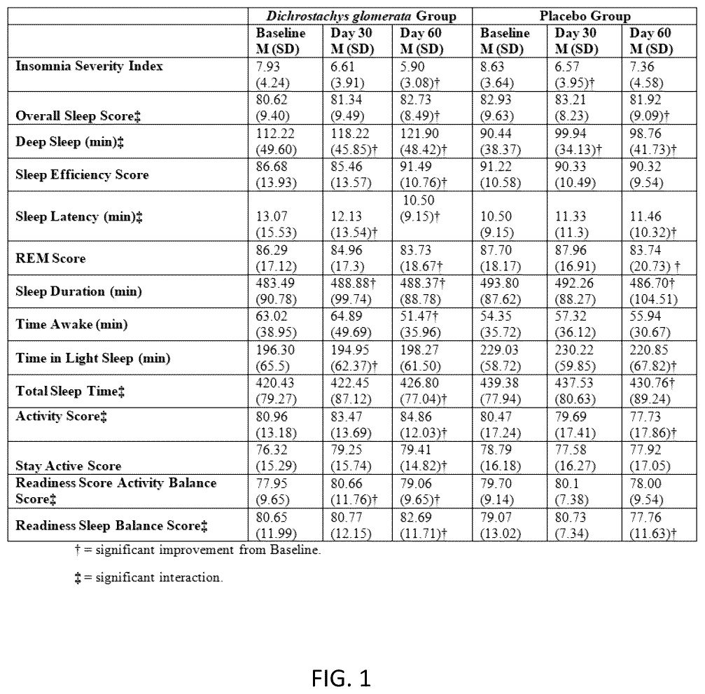

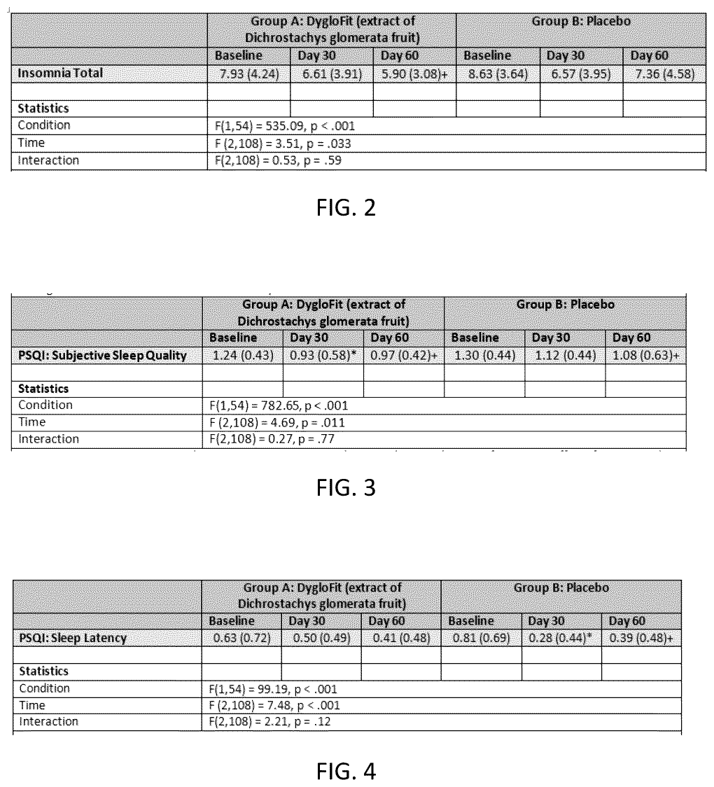

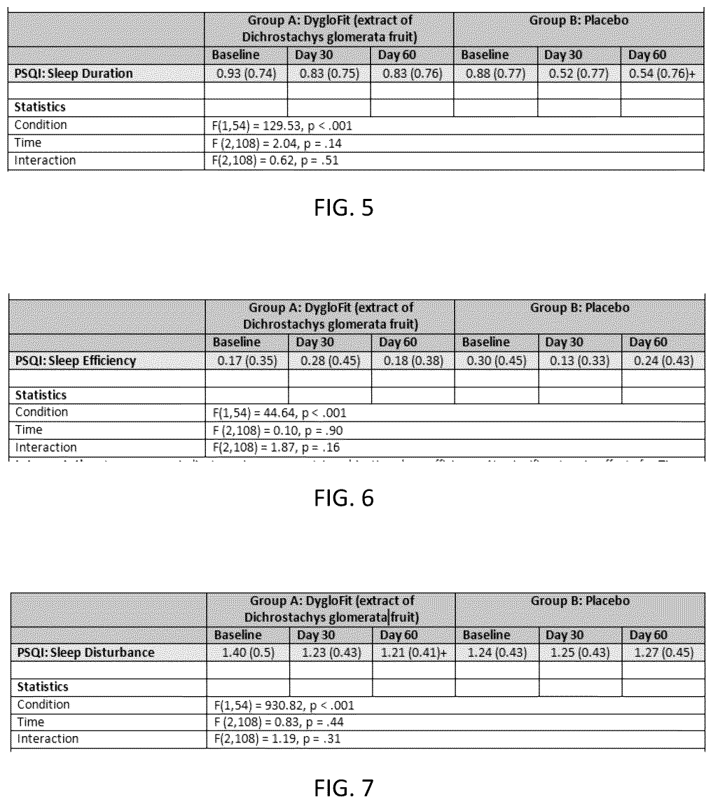

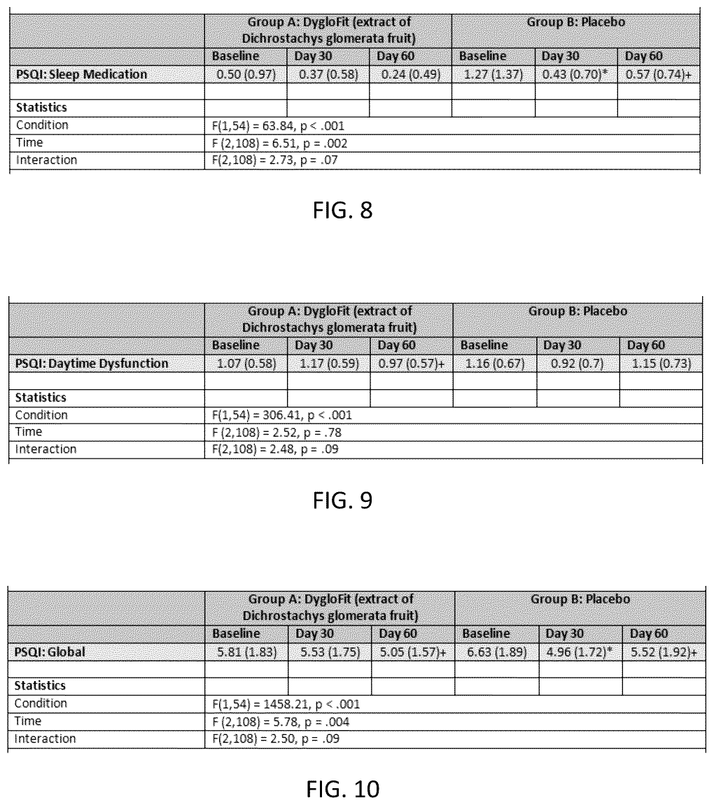

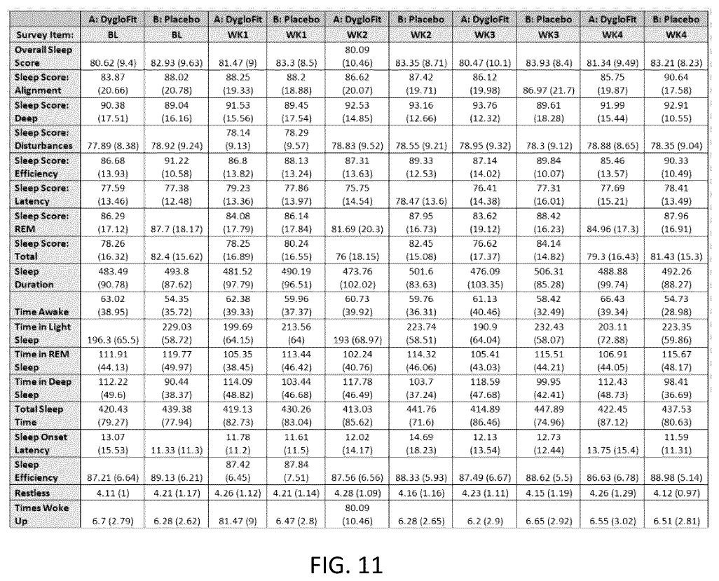

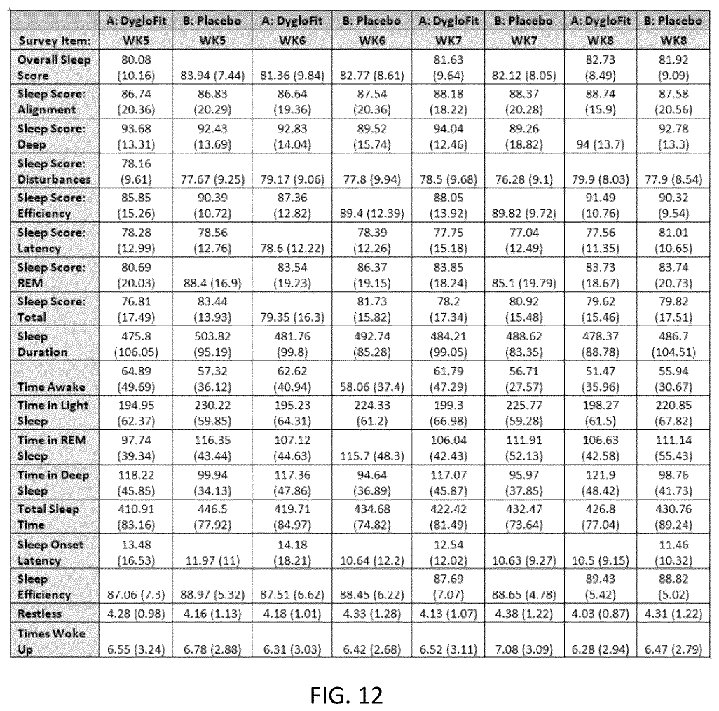

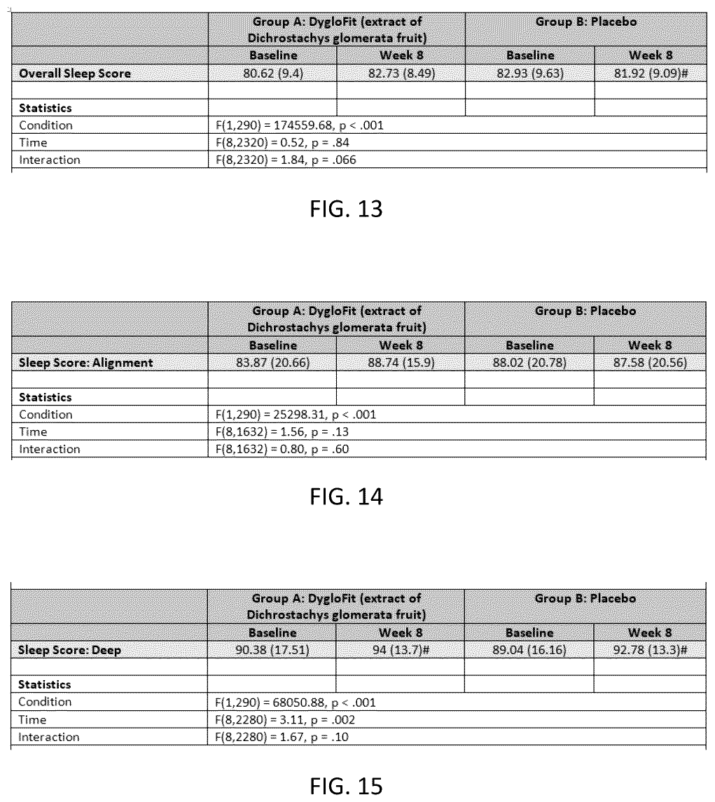

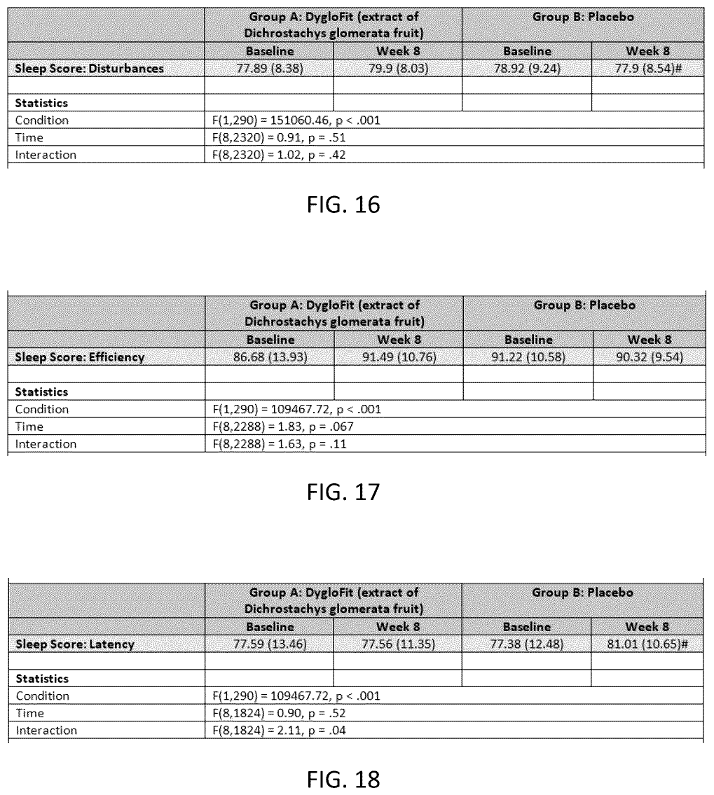

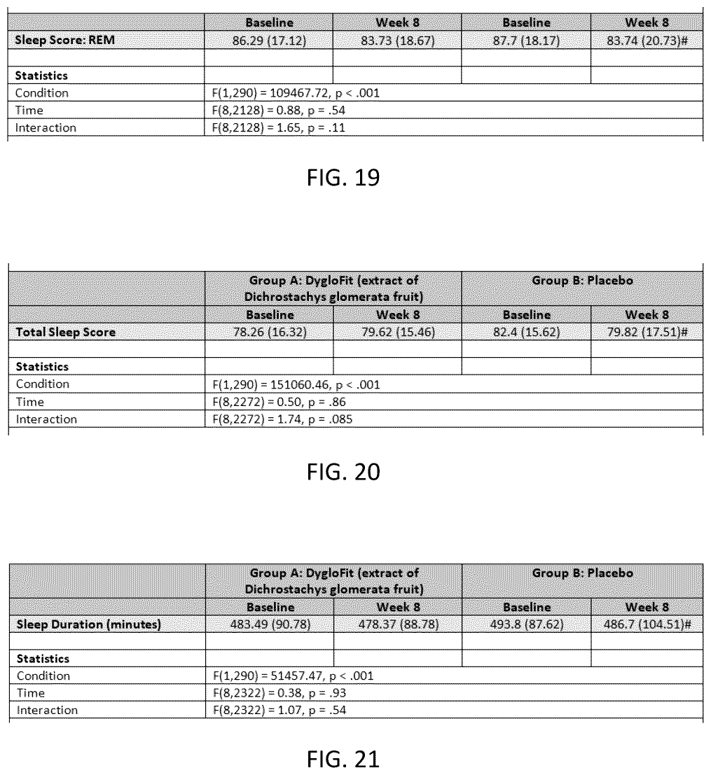

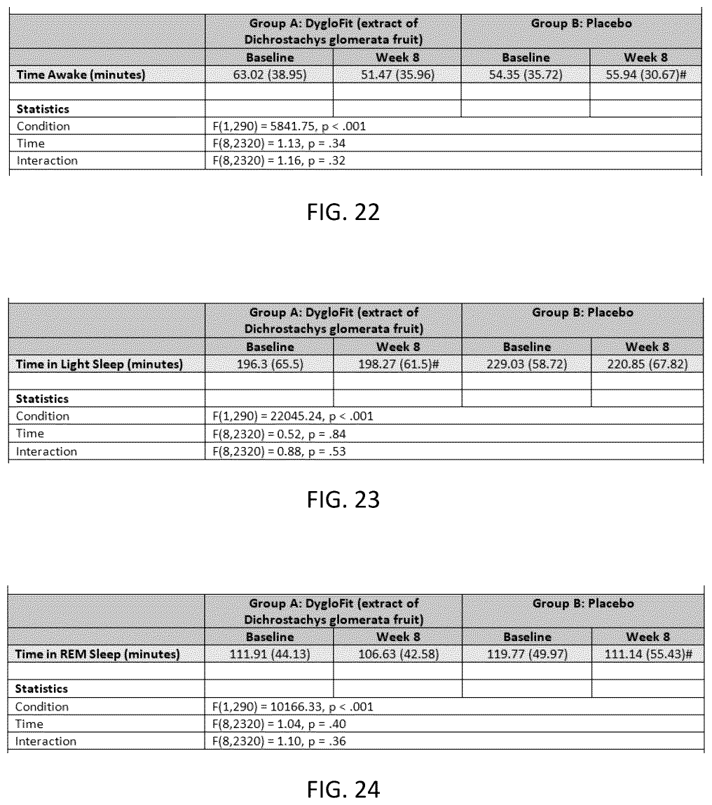

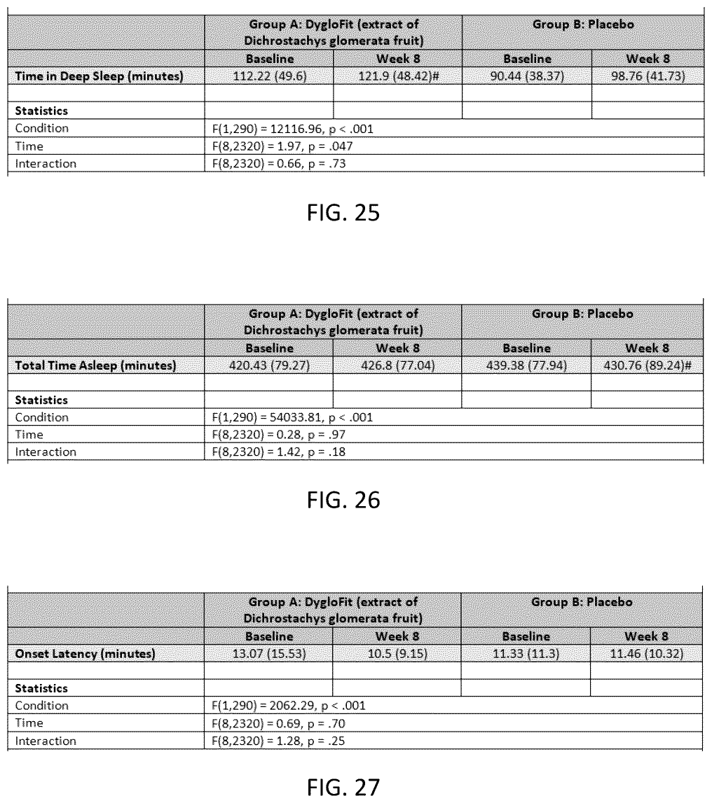

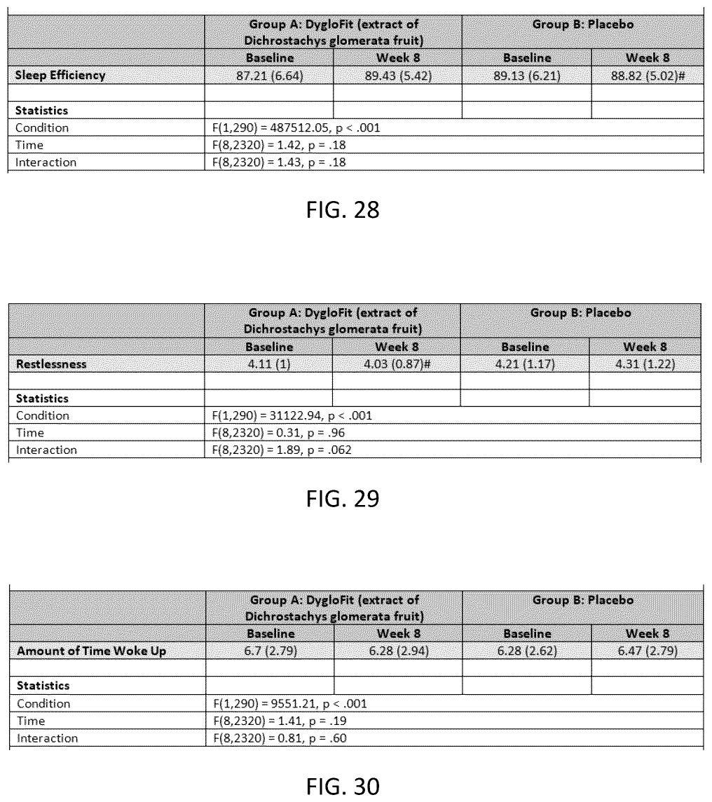

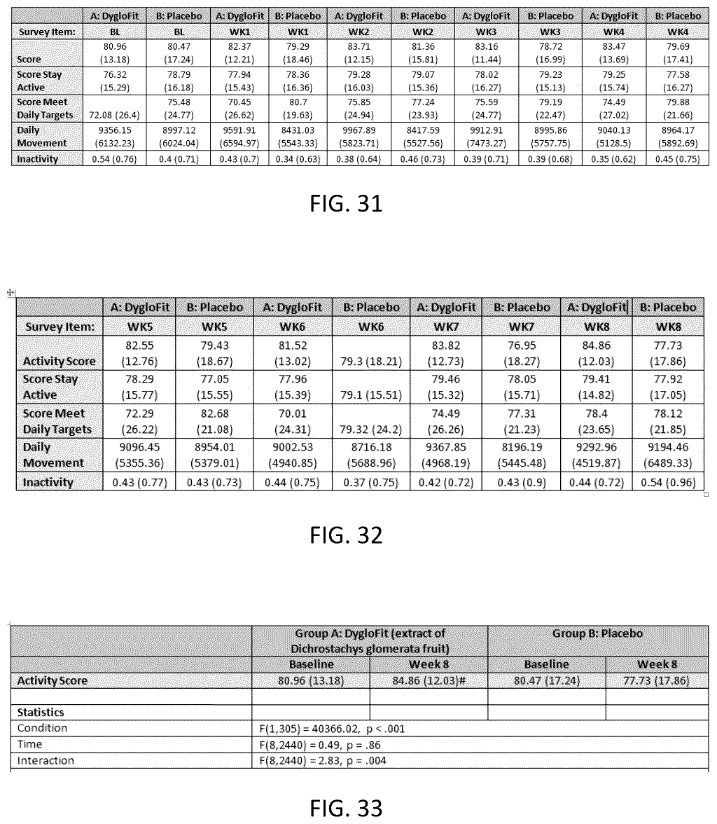

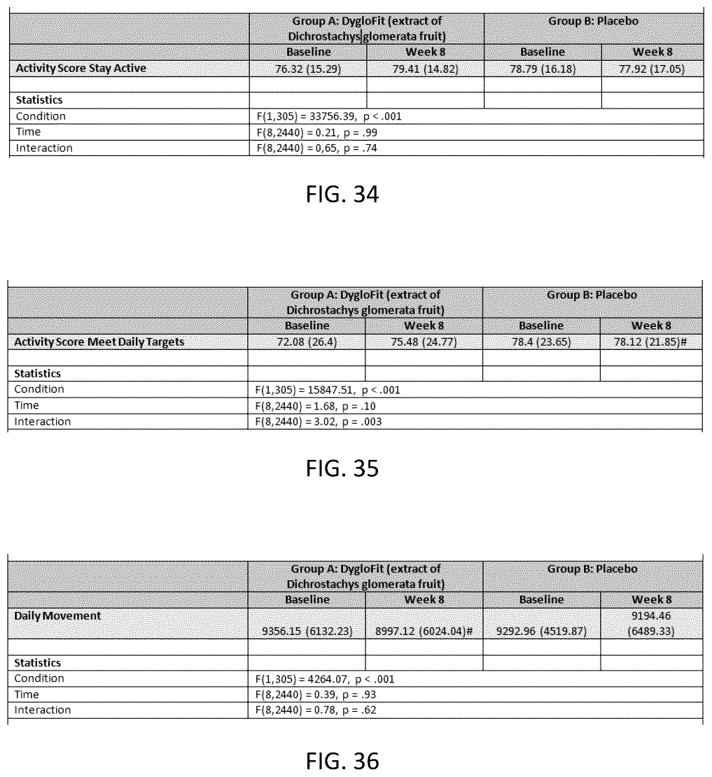

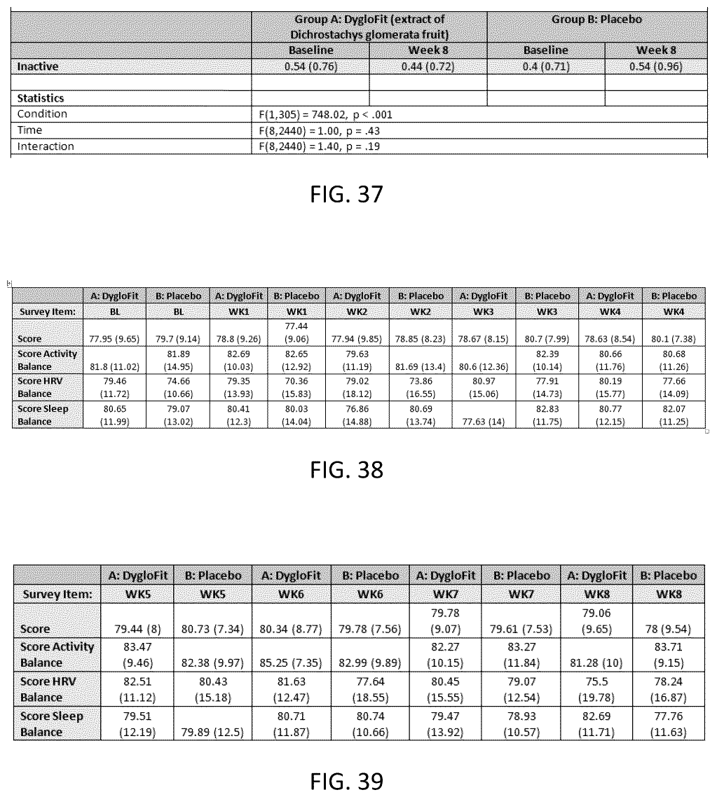

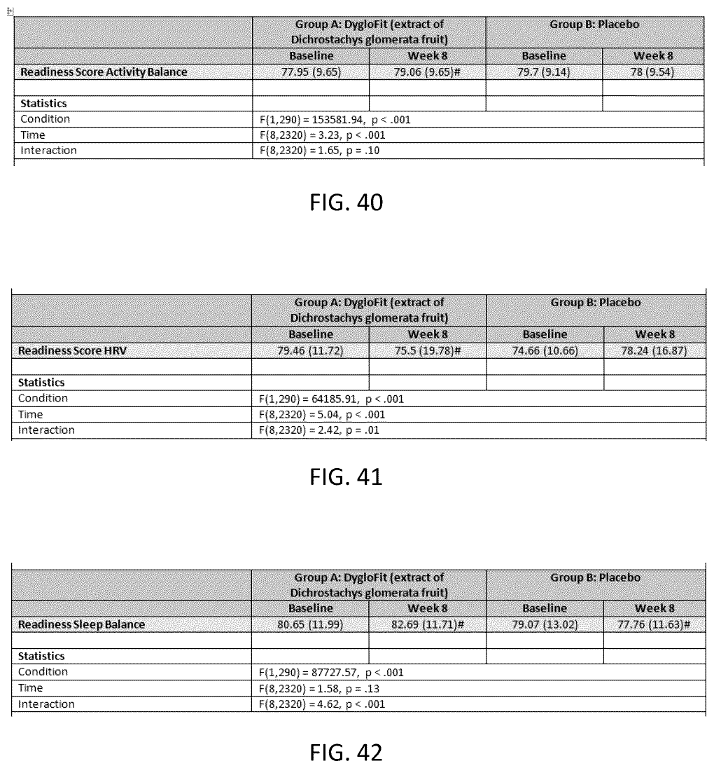

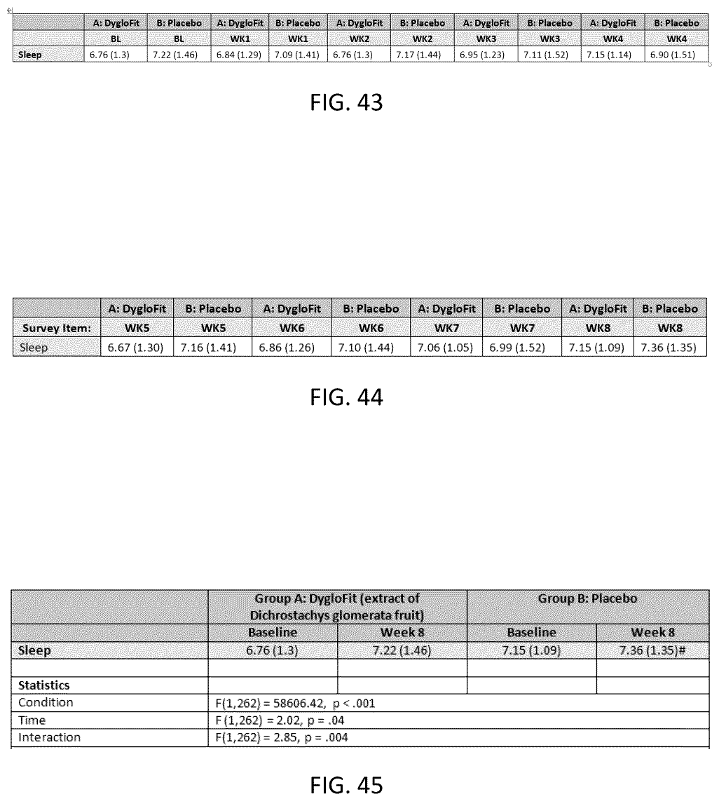

### DETAILED DESCRIPTION OF THE INVENTION

The following description and details are not to be taken in a limiting sense, but is made merely for the purpose of illustrating the general principles of the inventions disclosed herein. Various inventive features are described below that can each be used independently of one another or in combination with other features of the methods and compositions disclosed herein.

### Methods

Below are the details and methods of the double-blind, parallel treatment, stratified random, placebo-controlled study and experiments that were performed to determine the effects of the Dichrostachys glomerata composition that was provided or administered to mammals.

Participants: Participants were 56 adults (M age=44.50, age range=25 to 60 years, n=43 women).

Exclusion Criteria: Individuals meeting any of the following criteria were excluded from participation: (1) any metabolic or endocrine related dysregulation including but not limited to: diagnosis of type I or type II diabetes, liver, kidney, or pancreatic dysfunction; (2) history of sleep-affecting disorders; (3) highly stressful events within 4 weeks of baseline; (4) use of weight-influencing medications within 1 month of baseline; (5) use of Ca channel blockers, anxiolytics or SSRIs, no more than 5 times per month, and not within seven days of baseline; (6) unstable use of other medication; (7) current hormone therapy; (8) excessive alcohol consumption; (9) smoking; (10) elevated caffeine intake; (11) irregular sleep-inducing work schedules; (12) inability to engage in spontaneous physical activity; (13) metabolic disorder, a sleep disorder, or a psychiatric condition; (14) pregnancy, attempts at conception, or breastfeeding; (15) use of sleep/weight supplements or medications; (16) actively intermittent fasting, actively trying to lose weight, or have lost more than ±3 kg in previous 3 months; (17) moderate or severe obesity (BMI≥35), (18) clinical insomnia as determined by the Insomnia Severity Index, and (18) individuals deemed incompatible with the study protocol.

Study design: This study was approval by Sterling Institutional Review Board (10504) in compliance with the Declaration of Helsinki standards for ethical principles regarding human participant research and registered with ISRCTN registry (ISRCTN10099861).

This experiment and their results described herein were conducted in a double-blind, parallel treatment, stratified random, placebo-controlled manner. The independent variable was the Dichrostachys glomerata nutritional supplement. The dependent variables were sleep quality (primary outcomes), daytime activity and adverse events (secondary outcomes). Sample size power calculation indicated that 30 participants were needed in each group to achieve a power of 80% and alpha <0.05 (https://clincalc.com/stats/samplesize.aspx).

Procedures: Following preliminary screening, eligible participants provided Institutional Review Board approved informed consent prior to enrolment. Participants completed the Insomnia Severity Index on Day 0, Day 30, and Day 60. In addition, participants maintained a daily diary to document adherence and adverse events. Participants completed the self-report surveys via a SurveyMonkey link that was sent via email or text. Participants were instructed to maintain their habitual lifestyle patterns and refrain from introducing new exercise, diet, or health interventions during the study. Data were collected from approximately March 2023 to June 2023 and were stored electronically.

Intervention: A randomized double-blind placebo-controlled pilot trial design was employed, with participants randomly assigned to either the Dichrostachys glomerata group (hereafter, “DG”) or Placebo Control group (PG) for the duration of the two-month trial. A computer-based randomization via SPSS to automate the random assignment process was used. Participants were directed to consume 300 mg, 1×per day of the allocated substance. DyGlomera®, an aqueous ethanol extract of Dichrostachys glomerata fruit pods (standardized to Myricetin 1.6% and Luteolin 1.0%), that was supplied by Gateway Health Alliances, Inc (https://www.ghainc.com/; Fairfield, CA, USA). The manufacturing process was as follows: Dichrostachys glomerata fruit pods were extracted using aqueous ethanol. The resulting solution was concentrated and dried to yield DyGlomera®. The placebo was rice protein.

Trial Reporting: The Consolidated Standards of Reporting Trials (CONSORT, including reporting of harms) was used to report this trial.

Blinding: To ensure that all participants and researchers were unaware of the treatment assignments, Gateway Health Alliances provided the supplement and placebo labeled as either A or B. The supplement and placebo pills were identical in color, odor, and size. It should be appreciated that the compositions of the present disclosure can be provided in any form or delivery method, including but not limited to, pill, capsule, liquid, lozenge or tablet. At the conclusion of the study, immediately following the last assessment, the research team was unblinded. The participants were then unblinded and informed of their assigned condition.

Adherence: N=61 participants enrolled and consented and 56 completed the trial, representing an adherence rate of 92%. Two participants from the PG dropped out due to reasons unrelated to the study, and 3 participants (n=2 from the PG and n=1 from the DG) withdrew due to nonserious self-reported adverse events.

Statistical analysis: Data was analyzed for normality by examining skewness and kurtosis scores and using Shapiro-Wilk test and Q-Q plot. Outliers were characterized as data points that exceeded three interquartile ranges beyond 25th and 75th percentiles. However, no extreme outliers were observed. Continuous data were presented as Mean (SD) and analyzed using linear mixed model with Condition, and Time as fixed factors and subject as random factor. Statistical analyses were performed using Excel and Statistical Product and Service Solutions (SPSS) [version 28].

### Measures

Insomnia Severity Index: The Insomnia Severity Index is a 7-item self-report measure assessing symptoms of poor sleep. This index assesses sleep onset, sleep maintenance, early morning awakening problems; sleep dissatisfaction; interference of sleep difficulties with daytime functioning; whether sleep problems are noticed by others; and distress caused by sleep difficulties. The Insomnia Severity Index has excellent internal consistency (Cronbach alpha=0.91).¹⁷

Oura Ring. The Oura Ring is a novel, multisensory device that quantifies daily physical activity, night-time sleep duration, and estimates sleep stages, including REM (https://ouraring.com/). The ring also measures motion and body temperature. The Oura Ring uses physiological signals (a combination of motion, heart rate, heart rate variability, and pulse wave variability amplitude) in combination with sophisticated machine learning based methods to calculate deep, light and rapid-eye-movement (REM) sleep in addition to sleep/wake states. Rings are waterproof, made in ceramic, and come with a dedicated mobile App. They come in different sizes (US standard ring sizes 6-13) and weigh about 15 g with a battery life of about 3 days. The ring automatically connects via Bluetooth and transfers data to a mobile platform via the dedicated App. The Oura Ring has high validity in the assessment of nocturnal heart rate, heart rate variability, movement and sleep outcomes in healthy adults in their natural environment.¹⁸⁻²⁰

### Results

### Sleep Outcomes

For the Insomnia Severity Index, a significant main effect for Condition, F(1,54)=535.09, p<0.001, and Time, F(2,108)=3.51, p=0.03, and a nonsignificant interaction was evidenced, F(2,108)=0.53, p=0.59. Post hoc analyses revealed that the DG had nonsignificant improvements in insomnia symptoms from Baseline to Day 30, and significant improvements from Baseline to Day 60. The PG had a significant improvement from Baseline to Day 30, p<0.05, and a nonsignificant worsening of insomnia symptoms from Day 30 to Day 60.

For Overall Sleep Score, significant main effects for Condition, F(1,290)=174559.68, p<0.001, Time, F(8,2320)=0.52, p=0.84, and Interaction, F(8,2320)=1.84, p=0.05, were evidenced. Post hoc analyses indicated significant improvements in the Sleep Score from Baseline to Day 60 for the DG, p<0.05. In comparison, the PG had a significant decrease in the Sleep Score from Baseline to Day 60, p<0.05.

For Deep Sleep, significant main effect for Condition, F(1,290)=68050.88, p<0.001, and Time, F(8,2280)=3.11, p=0.002, and a nonsignificant interaction, F(8,2280)=1.67, p=0.10, were evidenced. Both the DG and PG's deep sleep improved significantly more from Baseline to Day 30 and Baseline to Day 60. Although the DG time in deep sleep improved more than the PG by Day 60, it was a nonsignificant interaction.

Sleep Efficiency represents the percentage of time spent asleep. Significant main effects for Condition, F(1,290)=109467.72, p<0.001, and Time, F(8,2288)=1.83, p=0.05, were evidenced. The interaction was nonsignificant, F(8,2288)=1.63, p=0.11. Post hoc analysis indicated a significant improvement from Baseline to Day 60 for the DG.

Sleep Latency is the amount of time that it takes to fall asleep at night. Significant main effects for Condition, F(1,290)=109467.72, p<0.001, and Interaction were found, F(8,1824)=2.11, p=0.04. The main effect for Time was nonsignificant, F(8,1824)=0.90, p=0.52. The DG sleep latency improved, while the PG worsened.

For REM sleep a significant main effect for Condition, F(1,290)=109467.72, p<0.001, was evidenced. The main effect for Time, F(8,2128)=0.88, p=0.54, and the Interaction, F(8,2128)=1.65, p=0.11, were nonsignificant. REM sleep decreased for both the DG and PG from Baseline to Day 60.

For Sleep Duration a higher score indicated a longer sleep duration. A significant main effect for Condition, F(1,290)=51457.47, p<0.001, was evidenced. The main effect for Time, F(8,2322)=0.38, p=0.93, and the Interaction, F(8,2322)=1.07, p=0.54, were nonsignificant. Post hoc analysis revealed a significant improvement in sleep duration from Baseline to Day 30 and Baseline to Day 60 for the DG. In comparison, the PG had a significant decrease in sleep duration from Baseline to Day 60.

Awake Time is the time spent awake in bed before and after falling asleep. Lower scores indicate less time awake during the night. A significant main effect for Condition, F(1,290)=5841.75, p<0.001, was evidenced. The main effect for Time, F(8,2320)=1.13, p=0.34, and the Interaction, F(8,2320)=1.16, p=0.32, were nonsignificant. The DG had a significant improvement in Time Awake from Baseline to Day 60, p<0.05, while the PG had a nonsignificant worsening of time awake at night at Day 30 and Day 60.

For Time in Light Sleep, significant main effect for Condition, F(1,290)=5841.75, p<0.001, was evidence. However, a nonsignificant main effect for Time, F(8,2320)=1.13, p=0.34, and Interaction, F(8,2320)=1.16, p=0.32, were found. The DG had a significant improvement in light sleep from Baseline to Day 30. The PG had a significant improvement from Baseline to Day 60.

Total sleep (minutes) reflects the amount of time spent in light, REM, and deep sleep. Significant main effects for Condition, F(1,290)=54033.81, p<0.001, and Interaction, F(8,2320)=1.42, p=0.05, were evidenced. The main effect for Time, F(8,2320)=0.28, p=0.97, was nonsignificant. The DG significantly improved in total sleep from Baseline to Day 60, while the PG significantly decreased in sleep from Baseline to Day 60, p<0.05.

For Sleep Readiness Balance, significant main effects for Condition, F(1,290)=87727.57, p<0.001, and the Interaction, F(8,2320)=4.62, p<0.001, were evidenced. The main effect for Time, F(8,2320)=1.58, p=0.13, was nonsignificant. Sleep readiness balanced improved significantly from Baseline to Day 60 for the DG, compared to a worsening for the PG.

### Activity Outcomes

For the Activity Score, a significant main effect for Condition, F(1,305)=40366.02, p<0.001, and interaction, F(8,2440)=2.83, p=0.004, were evidenced. The main effect for Time, F(8,2440)=0.49, p=0.86, was nonsignificant. The DG activity improved from Baseline to Day 60, and it decreased from Baseline to Day for the PG.

For Stay Active, significant main effect for Condition, F(1,305)=33756.39, p<0.001, was found. The main effect for Time, F(8,2440)=0.21, p=0.99, and the Interaction, F(8,2440)=0,65, p=0.74, were nonsignificant. The Stay Active scores improved over time for the DG and decreased for the PG.

For Activity Balance, significant main effects for Condition, F(1,290)=153581.94, p<0.001, and Time, F(8,2320)=3.23, p<0.001, were evidenced. The interaction was nonsignificant, F(8,2320)=1.65, p=0.10. Post hoc analyses revealed that Activity Balance improved significantly from Day 0 to Day 60 for the DG, and worsened for the PG.

Moderator analysis by gender revealed no gender effects, p's>0.05.

### Adverse Events

The supplement was well-tolerated and only one adverse event was reported for the DG compared to two adverse events reported for the CG. For the DG, the adverse event reported was gastrointestinal symptoms after taking the supplement. For the CG, the two adverse events reported were heart palpitations and a liver concern.

### Conclusion

The experiments that were performed and described herein, investigate the effectiveness of a standardized powder derived from Dichrostachys glomerata fruit pods on sleep quality for adults with nonclinical poor sleep quality using a randomized double-blind placebo-controlled design. The findings provide evidence that Dichrostachys glomerata supplementation improved and/or reduce insomnia, improved total sleep duration and quality, improved deep sleep duration and time in light sleep, improved sleep latency, improved the amount of time awake and improved sleep efficiency, readiness and balance in mammals as compared to the placebo. Interpretation of the results, experiment limitations and possibly implications are discussed below.

The DG displayed a notable trend toward improved self-reported Insomnia Severity Index scores, which reached significance by Day 60. In contrast, the PG had an initial significant improvement within the first 30 days, followed by a nonsignificant decrease in symptoms from Day 30 to Day 60. The Insomnia Severity Index encompasses various dimensions of sleep health, including sleep initiation and maintenance difficulties, early morning awakenings, satisfaction with sleep, impact on daily functioning, perception of sleep issues by others, and the distress associated with these difficulties.

Improvements in self-reported sleep for the DG were verified by the objective outcomes. The Oura Ring sleep parameters, including deep sleep, sleep latency, REM sleep, sleep efficiency, and light sleep provide a comprehensive understanding of sleep patterns and quality. The Dichrostachys glomerata supplementation demonstrated significant effects on various aspects of sleep and activity, highlighting its potential in promoting both improved sleep quality and daytime activity.

For example, the Overall Sleep Score, which measures overall objective sleep quality, the DG exhibited enhanced sleep quality over the 60-day study. Conversely, the PG experienced a decline in sleep quality. These findings underscore the divergent impact of this intervention on sleep quality, emphasizing the effectiveness of Dichrostachys glomerata supplementation in enhancing sleep outcomes within the DG. Importantly, the DG Overall Sleep Score of 82 fell within the range indicating good overall sleep quality.

Although the DG time in deep sleep improved more than the placebo group by Day 60, it was a nonsignificant interaction. Deep sleep refers to the physically restorative slow-wave sleep stage where bodily repair processes occur. The DG also improved in Time Awake from Baseline to Day 60, while the PG had a nonsignificant worsening of time awake at night at Day 30 and Day 60. Sleep efficiency, which represents the percentage of time spent asleep, improved for the DG from Baseline to Day 60. A sleep efficiency of 85% is a sign of peaceful and uninterrupted sleep.

From Day 0 to Day 60 the PG light sleep decreased, while the DG light sleep increased. Light sleep should account for between 45-55% of a healthy and natural sleep cycle. The DG significantly improved in Total Sleep from Baseline to Day 60, while the PG significantly decreased in sleep from Baseline to Day 60. Total Sleep reflects the amount of time spent in light, REM, and deep sleep. Finally, no improvements for the DG and PG were found for REM sleep which is associated with dreaming, memory consolidation, and creativity.

For daytime activity, the Stay Active scores improved over time for the DG and decreased for the PG. Stay Active measures how well the participant managed to avoid inactivity (sitting or standing still). For the Activity Score, the DG activity improved from Baseline to Day 60, and it decreased from Baseline to Day 60 for the PG. Activity is the time spent sitting, standing, or otherwise inactive. Inactive time does not include resting or sleep. Having five to eight hours or less of inactive time a day has a positive effect on the Activity Score. Moving regularly and avoiding long periods of inactivity helps adults stay healthy.

Similarly, Activity Balance improved significantly from Day 0 to Day 60 for the DG, and worsened for the PG. The Readiness Activity Balance scores measures how the activity level is affecting adult's readiness to perform. When the activity balance is optimal, it reflects adequate (and not excessive) activity. This boosts recovery and energy levels.

Finally, the Stay Active scores improved over time for the DG and decreased for the PG. Stay Active estimates the total daily inactive time while adults are awake. This contributor excludes when adults are naturally sedentary (e.g., asleep). To improve Stay Active contributor, adults should aim to keep inactive time under 8 hours each day.

The supplement was well-tolerated with only 1 mild adverse event reported in the DG. This finding expands genotoxicity results that found Dyglomera® was safe in an animal model.²¹ Different dietary supplements aimed at improving sleep quality are available on the market, and most have limited research supporting their efficacy. Research has found that amino acids, vitamin D, and melatonin supplements improve sleep quality.²² However, high heterogeneity and wide confidence levels plague this research, calling for a need for further clinical trials examining the effectiveness of dietary supplements. The results of this study provide data highlighting the effectiveness of Dyglomera® for improving and/or reducing insomnia, improving total sleep duration and quality, improving deep sleep duration and time in light sleep, improving sleep latency, improving the amount of time awake and improving sleep efficiency, readiness and balance in mammals.

### REFERENCES

1. Weinberg M K, Noble J M, Hammond T G. Sleep well feel well: An investigation into the protective value of sleep quality on subjective well-being. Aust J Psychol. 2016;68:91-97. doi: 10.1111/ajpy.12098.
2. Baglioni C, Nanovska S, Regen W, et al. Sleep and mental disorders: A meta-analysis of polysomnographic research. Psychol Bull. 2016;142(9):969-990. doi: 10.1037/bu10000053.
3. Suzuki M, Suzuki N, Sakurada K, Tsuchiya N, Ueno Y, Konta T. Insomnia symptoms and related factors in a community-based population: The Yamagata Cohort study. Heliyon. 2024;10(6):e28228. doi: 10.1016/j.heliyon.2024.e28228.
4. Gingerich S B, Seaverson E L D, Anderson D R. Association between sleep and productivity loss among 598,676 employees from multiple industries. Am J Health Promot. 2018;32:1091-4. doi: 10.1177/0890117117722517.
5. Ma Y, Liang L, Zheng F, Shi L, Zhong B, Xie W. Association between sleep duration and cognitive decline. JAMA Netw Open. 2020; 3:e2013573. doi: 10.1001/jamanetworkopen.2020.13573.
6. Ohayon M M. Epidemiological overview of sleep disorders in the general population. Sleep Med Res. 2011; 2:1-9. doi: 10.17241/smr.2011.2.1.1.
7. Kim J, Lee SL, Kang I, et al. Natural products from single plants as sleep aids: A systematic review. J Med Food. 2018; 21:433-44. doi: 10.1089/jmf.2017.4064.
8. Borrás S, Martínez-Solís I, Ríos JL. Medicinal plants for insomnia related to anxiety: An updated review. Planta Med. 2021;87(10-11):738-753. doi: 10.1055/a-1510-9826.
9. Djiazet S, Mezajoug Kenfack, L D, Linder M, Tchiégang C. An ethno-nutritional study on spices used in traditional foods of the Western Regions of Cameroon: the case of nah poh. J Ethn Foods. 019; 6(1). DOI: 10.1186/s42779-019-0030-6.
10. Djuissi N M, Ngoula F, Kouamo J, et al. Reproductive characteristics, serum metabolites, and oxidative status in female guinea pigs (Cavia porcellus) fed with ethanolic extract of Dichrostachys glomerata fruit. World Vet J. 2021; 11:66-72. doi: 10.54203/scil.2021.wvj9.
11. Etoundi C B, Kuaté D, Ngondi J L, Oben J. Anti-amylase, anti-lipase and antioxidant effects of aqueous extracts of some Cameroonian spices. J Nat Prod. 2010; 3:17.
12. Kuate D, Etoundi B C O, Soukontoua Y B, Ngondi J L, Oben J E. Antioxidant characteristics of Dichrostachys glomerata spice extracts. CYTA J Food. 2010;8:23-37. doi: 10.1080/19476330903129126.
13. Kuate D, Kegne A P N, Dakam W, Etoundi B C O, Paka G D. Effectiveness of Dichrostachys glomerata spice phenolics in reduction of oxidative stress associated with obesity and type 2 diabetes; A randomized, double-blind placebo-controlled clinical trial. J Food Res. 2013; 2:887-895.
14. Kuate D, Etoundi B C, Ngondi J L, Oben J E. Effects of Dichrostachys glomerata spice on cardiovascular diseases risk factors in normoglycemic and type 2 diabetic obese volunteers. Food Res Int. 2011;44:1197-1202. doi: 10.1016/j.foodres.2010.09.037.
15. Kuate D, Etoundi B C, Ngondi J L, Manan W A, Muda B W, Oben J E. Anti-inflammatory, anthropometric and lipomodulatory effects Dyglomera® (hydroethanolic extract of Dichrostachys glomerata) in obese patients with metabolic syndrome. Funct Foods Health Dis. 2013;3:416-427. doi: 10.31989/ffhd.v3i11.35.
16. Atrooz F, Salim S. Sleep deprivation, oxidative stress and inflammation. Adv Protein Chem Struct Biol. 2020; 119:309-336. doi: 10.1016/bs.apcsb.2019.03.001.
17. Bastien C H, Vallières A, Morin C M. Validation of the Insomnia Severity Index as an outcome measure for insomnia research. Sleep Med. 2001;2(4):297-307. doi: 10.1016/s1389-9457(00)00065-4.
18. de Zambotti, M, Rosas, L, Colrain, I M, Baker, F C. The sleep of the ring: Comparison of the OURA sleep tracker against polysomnography. Behav Sleep Med. 2019;17(2):124-136. doi: 10.1080/15402002.2017.1300587.
19. Kinnunen H, Rantanen A, Kenttä T, Koskimäki T. Feasible assessment of recovery and cardiovascular health: accuracy of nocturnal HR and HRV assessed via ring PPG in comparison to medical grade ECG. Physiol Meas. 2020; 7;41(4):04NT01. doi: 10.1088/1361-6579/ab840a.
20. Mehrabadi M A, Azimi I, Sarhaddi F et al., Sleep Tracking of a Commercially Available Smart Ring and Smartwatch Against Medical-Grade Actigraphy in Everyday Settings: Instrument Validation Study. JMIR Mhealth Uhealth. 2020; 8(10): e20465. doi: 10.2196/20465.
21. Kothari S C, Shivarudraiah P, Venkataramaiah S B, Gavara S, Arumugam N, Soni M G. Toxicologic evaluation of Dichrostachys glomerata extract: Subchronic study in rats and genotoxicity tests. Food Chem Toxicol. 2014;69:120-31. doi: 10.1016/j.fct.2014.03.045.
22. Chan V. Lo K. Efficacy of dietary supplements on improving sleep quality: a systematic review and meta-analysis. Postgrad Med J. 2022;98(1158):285-293. doi: 10.1136/postgradmedj-2020-139319.

What is claimed is:

1. A composition for improving sleep efficiency in a mammal, the composition comprising:
an effective amount of Dichrostachys glomerata;
wherein the composition is provided as an oral dosage unit in the form of a pill, capsule, liquid, lozenge or tablet.

2. The composition of claim 1, wherein the effective amount of Dichrostachys glomerata is 100 mg to 900 mg daily.

3. The composition of claim 1, wherein the Dichrostachys glomerata composition is an aqueous extract of Dichrostachys glomerata.

4. The composition of claim 1, wherein the Dichrostachys glomerata composition is an ethanol extract of Dichrostachys glomerata.

5. The composition of claim 1, wherein the Dichrostachys glomerata composition is comprised of Dichrostachys glomerata fruit pods.

6. The composition of claim 1, wherein the Dichrostachys glomerata composition is comprised of dried Dichrostachys glomerata fruit pods.

7. The composition of claim 1, wherein the composition is provided to a mammal for at least 30 days.

8. A composition for improving sleep readiness and balance in a mammal, the composition comprising:
an effective amount of Dichrostachys glomerata;
wherein the composition is provided as an oral dosage unit in the form of a pill, capsule, liquid, lozenge or tablet.

9. The composition of claim 8, wherein the effective amount of Dichrostachys glomerata is 100 mg to 900 mg daily.

10. The composition of claim 8, wherein the Dichrostachys glomerata composition is an aqueous extract of Dichrostachys glomerata.

11. The composition of claim 8, wherein the Dichrostachys glomerata composition is an ethanol extract of Dichrostachys glomerata.

12. The composition of claim 8, wherein the Dichrostachys glomerata composition is comprised of Dichrostachys glomerata fruit pods.

13. The composition of claim 8, wherein the Dichrostachys glomerata composition is comprised of dried Dichrostachys glomerata fruit pods.

14. The composition of claim 8, wherein the composition is provided to a mammal for at least 30 days.

15. A method of improving sleep efficiency, readiness and balance in a mammal, the method comprising:
providing a composition containing an effective amount of Dichrostachys glomerata to a mammal.

16. The method of claim 15, wherein the composition is provided as an oral dosage unit in the form of a pill, capsule, liquid, lozenge or tablet.

17. The method of claim 15, wherein the effective amount of Dichrostachys glomerata is 100 mg to 900 mg daily for at least 30 days.

18. The method of claim 15, wherein the Dichrostachys glomerata composition is an aqueous extract of Dichrostachys glomerata.

19. The method of claim 15, wherein the Dichrostachys glomerata composition is an ethanol extract of Dichrostachys glomerata.

20. The method of claim 15, wherein the Dichrostachys glomerata composition is comprised of Dichrostachys glomerata fruit pods.
